(19) United States
(12) Patent Application Publication
(10) Pub. No.: US 2013/0060189 A1
(43) Pub. Date: Mar. 7, 2013

(54) Multi-Needle Delivery System and Method

(71) Applicants: Carrie L. Herman, Mayer, MN (US); Benjamin Y. Arcand, Minneapolis, MN (US); Natalie A. Borgos, Lino Lakes, MN (US)

(72) Inventors: Carrie L. Herman, Mayer, MN (US); Benjamin Y. Arcand, Minneapolis, MN (US); Natalie A. Borgos, Lino Lakes, MN (US)

(73) Assignee: AMS RESEARCH CORPORATION, Minnetonka, MN (US)

(21) Appl. No.: 13/601,558

(22) Filed: Aug. 31, 2012

Related U.S. Application Data
(60) Provisional application No. 61/530,376, filed on Sep. 1, 2011.

(51) Int. Cl. A61M 5/00; A61M 25/10

(52) U.S. Cl. 604/96.01; 604/173

(57) ABSTRACT

A multi-needle injection system having a collapsible and expandable frame portion is provided. The system can be adapted for delivery trans-urethrally into the bladder, and expanded to touch or penetrate a wall portion of the bladder. A plurality of positioned needles can inject at multiple points into the bladder wall simultaneously to introduce a therapeutic or treatment substance.

### PRIORITY

The Application claims priority to and the benefit of U.S. Provisional Application No. 61/530,376, filed Sept. 1, 2011, which is incorporated herein by reference in its entirety.

### FIELD OF THE INVENTION

The invention relates generally to urinary disorder treatment tools and methods and, more particularly, to a device, system and method of introducing and injecting a treatment substance to a patient's bladder tissue using an expandable frame and needle structure.

### BACKGROUND OF THE INVENTION

Urinary incontinence is a significant health concern worldwide. For example, lower urinary tract disorders affect the quality of life of millions of men and women in the United States every year. These disorders include overactive bladder. Overactive bladder is a treatable medical condition that is estimated to affect 17 to 20 million people in the United States. Current treatments for overactive bladder include medication, diet modification, programs in bladder training, electrical stimulation, and surgery. There is a continuing desire to provide additional treatment options that can be used as an alternative to, or in conjunction with, the current treatment options.

### SUMMARY OF THE INVENTION

The invention relates generally to devices and method for delivering therapeutic or treatment substances, such as botox, stem cells, adipose, agents, drugs or the like to the wall of the bladder for treatment of urinary tract disorders, including over active bladder. In certain embodiments, a low profile catheter and/or frame portion is provided that can be delivered trans-urethrally into the bladder, expanded to touch or penetrate a portion (e.g., wall) of the bladder at multiple points. A plurality of positioned needles can inject at multiple points into the bladder wall simultaneously to introduce the therapeutic or treatment substance.

The catheter can include frame to provide a plurality of injection micro-needles in a protected delivery configuration. The needles are deployed into the bladder wall upon expansion of the frame. The can be constructed of a shape memory material, such as Nitinol. Other embodiments can be constructed of elastic materials, polymers or like materials. The frame can include features that interface with short needles attached to polymer micro-tubes. The micro-tubes can be flexible and are in operable fluid communication with the needles and the catheter to facilitate fluid transfer. Upon deployment, the frame shape holding the needles deforms to rotate the needle tips a measurable amount in a generally outward or radial direction to facilitate engagement with and injection into the bladder wall. Withdrawal of the frame back into the delivery configuration brings the needles back to a protection position within the device to avoid inadvertent tissue puncturing or damage.

Expansion of the shape memory frame can be accomplished by mechanical means, electronic means, or other known devices and techniques. In one embodiment, a mechanism or technique compresses the frame in the axial direction to cause the frame to bend inwards. Further, the frame can be heat set in a “deployed” configuration and compressed into a sheath or cannula for delivery. Release or deployment of the frame from the sheath allows expansion of the device into the bladder. In another embodiment, the frame expansion can be facilitated by an inflatable balloon or like expanding element on the interior or exterior of the frame. Expansion of the element can correspondingly expand the frame and the needles against the bladder wall.

The device can be constructed such that the frame itself serves as the needle body, including the lumens or conduits for transferring and injecting fluid through the catheter, frame and out the needles, without distinct polymer or like micro-tubes.

A key advantage of this system and device is to provide the use of multiple injection needles for a fast “single” injection procedure that can deliver a known amount of injectate in a defined and even pattern into the bladder wall for an effective and targeted treatment. Further, the use of this needle configuration as the injection method allows for the injection of fragile live cell therapeutics that would otherwise be destroyed by a hydraulic or jet injection technique and procedure.

### BRIEF DESCRIPTION OF THE DRAWINGS

FIG. 1 is a schematic front view of a selectively collapsible and expandable multi-needle injection system in an expanded configuration, in accordance with embodiments of the present invention.

FIG. 2 is a schematic front view of a selectively collapsible and expandable multi-needle injection system in a collapsed or partially collapsed configuration, in accordance with embodiments of the present invention.

FIG. 3 is a partial schematic side view of a selectively collapsible and expandable multi-needle injection system in a collapsed or partially collapsed configuration, in accordance with embodiments of the present invention.

FIG. 4 is a partial schematic front view of a selectively collapsible and expandable multi-needle injection system in a collapsed or partially collapsed configuration, in accordance with embodiments of the present invention.

FIG. 5 is a partial schematic front view of a selectively collapsible and expandable multi-needle injection system in an expanded or partially expanded configuration, in accordance with embodiments of the present invention.

FIG. 6 is a partial schematic side view of a selectively collapsible and expandable multi-needle injection system in a collapsed configuration within a delivery sheath or cannula, in accordance with embodiments of the present invention.

FIG. 7 is a schematic side view of a selectively collapsible and expandable multi-needle injection system having a balloon or inflation element in a collapsed configuration, in accordance with embodiments of the present invention.

FIG. 8 is a schematic top view of a selectively collapsible and expandable multi-needle injection system having a balloon or inflation element, in accordance with embodiments of the present invention.

FIG. 9 is a schematic top view of a selectively collapsible and expandable multi-needle injection system having a balloon or inflation element in an expanded configuration, in accordance with embodiments of the present invention.

FIG. 10 is a partial schematic front view of a selectively collapsible and expandable multi-needle injection system expanding for engagement with target tissue, in accordance with embodiments of the present invention.

### DETAILED DESCRIPTION OF PREFERRED EMBODIMENTS

The present invention is directed to devices, instruments, assemblies and methods for delivering injectables to the wall or inner lining of the bladder, for treatment of urinary tract disorders, including over active bladder. Embodiments can insert the devices, frame and needle construct into other pelvic lumens, cavities and tissue zones to effectuate like treatment via injectables.

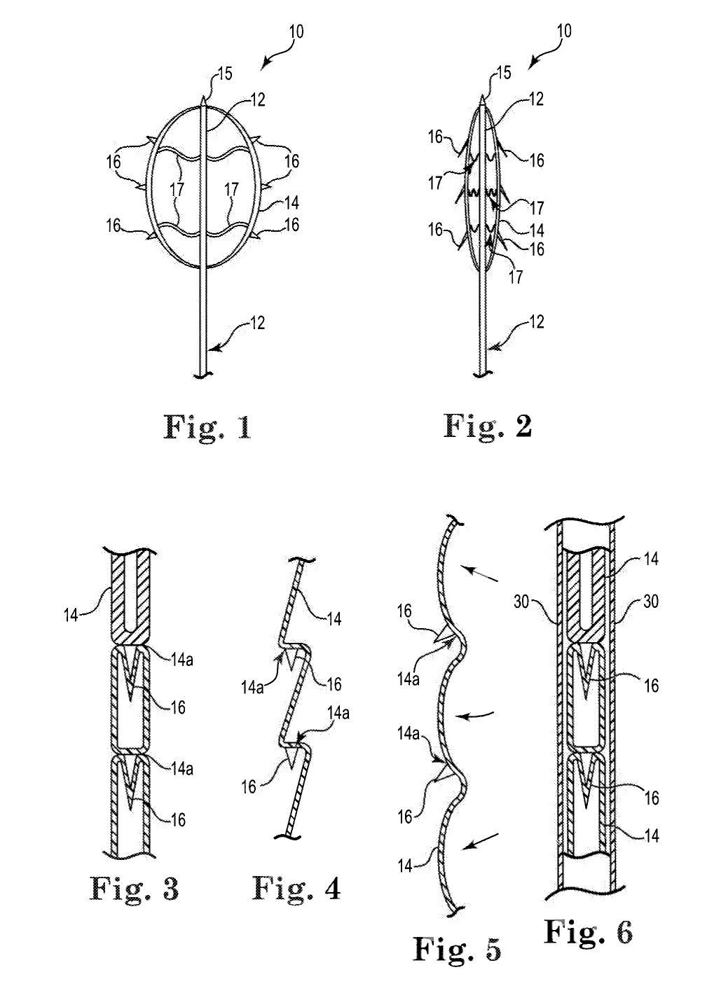

Referring generally to FIGS. 1-10, embodiments of a needle injection device and system 10 are provided. The device 10 can include a delivery catheter 12. Further, the device 10 or catheter 12 can include a shape memory, hinged, or like frame 14 to hold or provide a plurality of injection needles 16 in a protected or collapsed delivery configuration. The needles 16 can be micro-needles deployed into the bladder wall W upon expansion of the frame 14, and include one or more fluid ejection apertures at or proximate the tip. The frame 14 can be constructed of elastic materials, shape-memory Nitinol, stainless steel or the like. Other embodiments can be constructed of polymers or like materials capable of collapsing, or retracting, and then expanding upon deployment.

The frame 14 can include features that interface with short needles 16 attached to polymer micro-tubes 17. The micro-tubes 17 can be flexible and in operable fluid communication with the needles 16 and the catheter 12 to facilitate fluid transfer. The locations 14a where the needles 16 are attached with the frame 14 can hold the needles in a protected configuration by the shape of the frame in those locales. For instance, as shown in FIGS. 3-4, the needles 16 can be configured to generally face down, or within an indent or pocket of the frame 14, or can be otherwise protected during deployment through body lumens and tissue.

Upon deployment, the frame 14 shape holding the needles deforms to rotate the needle tips 16 in a generally outward or radial direction to facilitate penetration and injection into the bladder wall W, as shown in FIGS. 2, 5 and 9-10. Collapsing the frame 14 back into the delivery configuration brings the needles 16 back to a protection position within the device to avoid inadvertent tissue puncturing or damage during deployment or other internal movement. The targeted tissue can be limited to a portion of the interior bladder wall, or the device 10 can be expanded to contact more than one wall portion, depending on the size and configuration of the device 10 in the expanded state. For instance, with certain embodiments, the device 10 can expand to generally the same size as the interior of the bladder to provide multiple contact and injection sites along multiple interior wall portions of the bladder.

Injectables or injectates for use with the needle constructs described herein can include botox, stem cells, biologics, agents, drugs or the like therapeutic or treatment substances for injection into the wall of the bladder for treatment of urinary tract disorders, including an over active bladder. The injectate can serve to bolster, strengthen, heal or otherwise treat the target tissue site. The needles 16 and other structures and features of the system 10, including the frame 14, micro-tubing 17 and delivery catheter 12, can include communication lumens, conduits and the like, in operable fluid communication, to facilitate fluid traversal and eventual injection from the catheter 12 and out through the needles 16 and into the target tissue.

In various embodiments, portions of or all of the frame 14 members can be generally hollow in the form of tubing, or micro-tubing, (e.g., metal or polymer) to define the needle delivery construct in fluid communication with the needles 16 and the catheter portion 12 to facilitate transfer and injection of the treatment substance into the tissue wall, e.g., without separate polymer or like micro-tubing.

Expansion of the frame 14 can be accomplished by mechanical means, electronic means, or other known mechanisms, devices and techniques. In one embodiment, a mechanism or technique compresses the frame 14 in the axial direction to cause the frame 14 to bend or collapse inwards (e.g., FIG. 2). In certain embodiments, this can be accomplished by moving the catheter 12 or a like member against the tip 15 (which can also serve as a needle 16) or other portion of the frame 14 to cause the frame 14 to collapse. Likewise, the catheter 12 or like member can be pulled back or retracted to expand the frame 14. In other embodiments, pushing on a mechanism or member, such as catheter 12, operably connected to a portion of the frame 14, can retract or collapse the frame 14 for deployment.

Further, the frame 14 can be heat set or otherwise pre-formed in a “deployed” configuration and compressed through or within a sheath or delivery needle or cannula 30 for introduction to the target site during the procedure, as shown in FIG. 6. Release or deployment of the frame 14 from the sheath or cannula 30 allows expansion of the device 10 into the bladder, and into contact with the tissue wall.

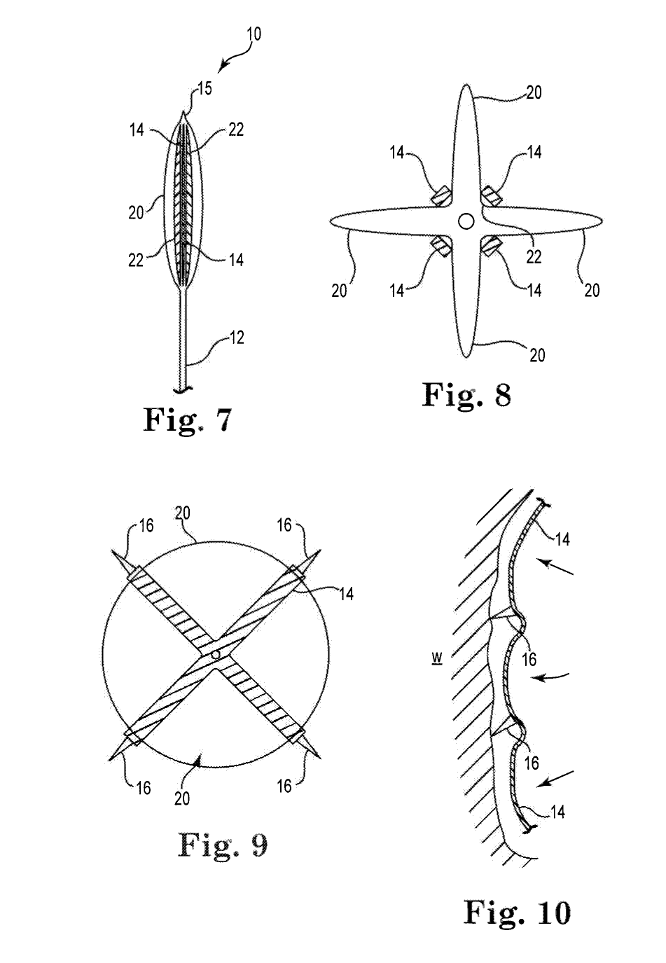

In another embodiment, as shown in FIGS. 7-9, the frame 14 expansion can be facilitated by an inflatable balloon or like element 20 provided on or along the interior or exterior of the frame 14. Expansion of the element 20 can correspondingly expand the frame 14 and the needles 16 against the target injection site, such as the bladder wall W. The frame 14 can be attached to the element 20 via adhesives, clips, interweaving or sewing, sutures, bonding, or a myriad of other structures or techniques.

Moreover, portions of the frame 14 can be contained or confined within the pleats or folds 22 of the element 20 when the element 20 and/or frame 14 are collapsed, as shown in FIG. 7. This can serve to facilitate deployment and protect from unwanted tissue puncturing or damage during introduction of the device 10. The fluid conduits or micro-tubes 17 can be included within the element, or the frame 14 members can serve as the fluid transfer conduit as described herein. In other embodiments, the interior of the element 20 can be simply configured to hold fluid for injection, e.g., in fluid communication with the catheter 12 and the needles 14 and without additional micro-tubes.

In other embodiments, the element 20 itself can include the needles 16 integrated or provided along portions of the element 20 for selective expansion and collapsing without the use of the frame 14. Again, the interior of the element 20 can contain the injectate for delivery to the tissue wall via the needles 16 in fluid communication with the interior cavity of the element 20, or one or more micro-tubes 17 can be employed.

The element 20 and needles 16 can take on any suitable size and shape depending on the particular treatment application and procedure. In one embodiment, the element 20, or the overall fluid system of the device 10, is able to deliver between 20 ml and 40 ml (e.g., 30 ml) of fluid, and the micro needle inner diameter is between 0.2 mm and 0.5 mm (e.g., 0.337 mm).

In use, the frame 14 and/or catheter 12 can be delivered trans-urethrally into the bladder, and expanded such that the needles 16 touch or at least partially penetrate a portion (e.g., wall W) of the bladder at multiple points. The plurality of positioned needles 16 can inject at multiple points into the bladder wall W simultaneously to introduce the therapeutic or treatment substance.

It is understood that the present invention can be used to deliver a variety of treatment fluids to patient tissue. Moreover, a variety of introduction devices, injection techniques and structures, substances or fluid, such as those disclosed and taught in U.S. Patent Application Publication Nos. 2011/0008299 and 2012/0101472, can be employed in conjunction with embodiments of the present invention, and therefore each of the above-referenced publications are incorporated herein by reference in their entirety.

The balloon, frame and needles can be comprised of any bio-compatible and safe material. The micro needle components can be safe for implantation or short term tissue contact, depending on the particular application and procedure.

All patents, patent applications, and publications cited herein are hereby incorporated by reference in their entirety as if individually incorporated, and include those references incorporated within the identified patents, patent applications and publications.

Obviously, numerous modifications and variations of the present invention are possible in light of the teachings herein. It is therefore to be understood that within the scope of the appended claims, the invention may be practiced other than as specifically described herein.

1. A multi-needle injection device, comprising:
a catheter portion having a catheter lumen;
a frame portion having a plurality of frame members adapted to collapse to a deployment state and expand to an injection state; and
a plurality of injection needle tips provided along one or more of the frame members in fluid communication with the catheter and adapted to inject a treatment substance into target tissue.

2. The device of claim 1, wherein one or more of the plurality of frame members is constructed of a shape memory material.

3. The device of claim 2, wherein the shape memory material is Nitinol.

4. The device of claim 1, further including a plurality of tubing members in fluid communication with the needle tips and the catheter.

5. The device of claim 4, wherein the plurality of tubing members are generally contained within the frame portion.

6. The device of claim 1, wherein the target tissue is an interior wall of the bladder.

7. The device of claim 1, wherein one or more of the frame portions is generally hollow and in fluid communication with the catheter portion and one or more of the plurality of injection needle tips.

8. The device of claim 1, further including an inflatable element.

9. The device of claim 8, wherein the inflatable element is an inflatable balloon element.

10. The device of claim 8, wherein the frame portion is provided along a portion of the inflatable element.

11. The device of claim 10, wherein the inflatable element is in fluid communication with the catheter portion and the plurality of injection needle tips.

12. The device of claim 1, further including a delivery sheath adapted to contain at least the frame portion in the deployment state such that the frame portion is expanded to the injection state upon removal from the delivery sheath.

13. A multi-needle injection device, comprising:
a catheter portion;
a frame portion adapted to collapse to a deployment state and expand to an injection state;
a plurality of injection needle tips provided along the frame portion and in fluid communication with the catheter, the plurality of injection needle tips adapted to inject a treatment substance into target tissue; and
further including a plurality of conduits in fluid communication with the needle tips and the catheter.

14. The device of claim 13, wherein the frame portion is constructed of a shape memory material.

15. The device of claim 14, wherein the shape memory material is Nitinol.

16. The device of claim 13, wherein the target tissue is an interior wall of the bladder.

17. The device of claim 13, wherein the plurality of conduits are provided within the frame portion.

18. The device of claim 13, wherein the plurality of conduits are polymer tubing members.

19. The device of claim 13, further including an inflatable element adapted to carry the frame portion.

20. The device of claim 19, wherein the inflatable element is in fluid communication with the catheter portion and the plurality of injection needle tips.
